(19) United States
(12) Patent Application Publication
(10) Pub. No.: US 2021/0330840 A1
(43) Pub. Date: Oct. 28, 2021

(54) SYSTEMS AND METHODS FOR REDUCING MICROBIAL AND/OR VIRAL LOADS ON EQUIPMENT USING OZONE

(71) Applicant: The Texas A&M University System, College Station, TX (US)

(72) Inventors: David Staack, College Station, TX (US); Suresh D. Pillai, College Station, TX (US); Matt Pharr, College Station, TX (US); Kavita Rathore, Bryan, TX (US); John Lassalle, College Station, TX (US); Matthew L. Burnette, Bryan, TX (US); Min Huang, College Station, TX (US); Md Kamrul Hassan, College Station, TX (US)

(73) Assignee: The Texas A&M University System, College Station, TX (US)

(21) Appl. No.: 17/243,344

(22) Filed: Apr. 28, 2021

Related U.S. Application Data
(60) Provisional application No. 63/016,340, filed on Apr. 28, 2020.

(51) Int. Cl. A61L 2/20

(52) U.S. Cl. CPC A61L 2/202; A61L 2202/15; A61L 2202/121

(57) ABSTRACT

A system for reducing a microbial and/or viral load on equipment using ozone includes a container including having an open configuration to provide access to an interior of the container and a closed configuration to seal the interior from an environment external the container, and wherein the interior includes a receptive region to receive the equipment, a circulation fan positioned in the interior of the container, and one or more ozone generators positioned in the interior of the container and configured to generate ozone upon activation, wherein the circulation fan is configured to provide an airflow including ozone generated by the one or more ozone generators and directed along a flowpath extending into the receptive region of the interior of the container.

### CROSS-REFERENCE TO RELATED APPLICATIONS

This application claims benefit of U.S. provisional patent application Ser. No. 63/016,340 filed Apr. 28, 2020, and entitled “Plasma Generated Ozone and Reactive Oxygen Species for Point of Use PPE Decontamination System,” which is hereby incorporated herein by reference in its entirety for all purposes.

### STATEMENT REGARDING FEDERALLY SPONSORED RESEARCH OR DEVELOPMENT

Not applicable.

### BACKGROUND

Techniques employing reactive oxygen species and other reactive materials have been investigated in the field of decontamination and bioburden reduction for various types of equipment. For example, it has been found that ionized gas, ultraviolet (UV) radiation, oxygen species (O, O3, and P2*), and oxygen-containing radials (e.g., OH* and NO*) may inactivate certain microorganisms present on the surfaces of the equipment to be decontaminated. Particularly, UV photons and highly reactive short-lived species (e.g., accelerated ions and electrons, uncharged particles such as excited atoms, molecules, and radicals) all participate in various inactivation mechanisms. Reactive species, including reactive oxygen species (ROSs), may be generated as a plasma from a suitable generator such as a dielectric barrier discharge (DBD) reactor.

### BRIEF SUMMARY OF THE DISCLOSURE

An embodiment of a system for reducing a microbial and/or viral load on equipment using ozone comprises a container comprising having an open configuration to provide access to an interior of the container and a closed configuration to seal the interior from an environment external the container, and wherein the interior comprises a receptive region to receive the equipment, a circulation fan positioned in the interior of the container, and one or more ozone generators positioned in the interior of the container and configured to generate ozone upon activation, wherein the circulation fan is configured to provide an airflow comprising ozone generated by the one or more ozone generators and directed along a flowpath extending into the receptive region of the interior of the container. In some embodiments, the container comprises a road-transportable trailer comprising a plurality of wheels. In some embodiments, the system further comprises an electrical generator supported on the trailer and configured to power the one or more ozone generators and the circulation fan. In certain embodiments, the container comprises a human-portable glovebox. In certain embodiments, the receptive region is spaced from the one or more ozone generators by a predefined distance. In some embodiments, the circulation fan is configured to provide the airflow at a flowrate such that a predefined diffusion time is elapsed before the ozone reaches the receptive region. In some embodiments, the diffusion time is between five seconds and 90 seconds. In some embodiments, the system further comprises a humidifier configured to maintain a humidity in the interior of the container in a predefined humidity range between 75% relative humidity (RH) and 95% RH. In certain embodiments, the one or more ozone generators are configured to effect at least a 3-log reduction in a microbial or viral load on the equipment in response to exposing the equipment to a dose of between 450 parts per million minutes (ppm-min) and 650 ppm-min. In certain embodiments, the one or more ozone generators are configured to effect at least a 6-log reduction in a microbial or viral load on the equipment in response to exposing the equipment to a dose of between 1450 parts per million minutes (ppm-min) and 1550 ppm-min. In some embodiments, a ratio of a distance between the one or more ozone generators and the receptive region, and a flowrate to which the circulation fan is configured to provide is between five and 90. In some embodiments, a ratio of a distance between the one or more ozone generators and the receptive region, and a flowrate to which the circulation fan is configured to provide is between 20 and 45. In some embodiments, the system further comprises a wire shelf positioned in the interior of the container and configured to receive the equipment.

An embodiment of a method for reducing a microbial and/or viral load on equipment using ozone comprises (a) positioning the equipment in a receptive region within an interior of a container, (b) sealing the interior of the container from an environment external the container, (c) activating one or more ozone generators positioned in the interior of the container to generate ozone, and (d) operating a circulation fan positioned in the interior of the container to provide an airflow comprising the ozone generated by the one or more ozone generators and directed along a flowpath extending into the receptive region of the interior of the container. In some embodiments, the equipment comprises personal protective equipment (PPE). In some embodiments, the container comprises a road-transportable trailer comprising a plurality of wheels. In certain embodiments, (d) comprises effecting at least a 3-log reduction in a microbial or viral load on the equipment in response to exposing the equipment to an ozone dose of between 450 parts per million minutes (ppm-min) and 650 ppm-min. In certain embodiments, (d) comprises effecting at least a 6-log reduction in a microbial or viral load on the equipment in response to exposing the equipment to an ozone dose of between 1450 parts per million minutes (ppm-min) and 1550 ppm-min. In some embodiments, a ratio of a distance between the one or more ozone generators and the receptive region, and a flowrate to which the circulation fan is configured to provide is between five and 90. In some embodiments, a ratio of a distance between the one or more ozone generators and the receptive region, and a flowrate to which the circulation fan is configured to provide is between 20 and 45. In certain embodiments, the method comprises (e) maintaining a humidity in the interior of the container in a predefined humidity range between 75% relative humidity (RH) and 95% RH.

### BRIEF DESCRIPTION OF THE DRAWINGS

For a detailed description of exemplary embodiments of the disclosure, reference will now be made to the accompanying drawings in which:

FIG. 1 is a side view of an embodiment of a system in accordance with principles described herein for reducing a microbial and/or viral load on equipment comprising a polymeric material using ozone and/or other ROSs;

FIG. 2 is a front view of the system of FIG. 1;

FIG. 3 is a rear view of the system of FIG. 1;

FIG. 4 is a side cross-sectional view of the system of FIG. 1;

FIG. 5 is a graph illustrating ozone concentration over time;

FIG. 6 is a graph illustrating humidity and temperature over time;

FIG. 7 is a side cross-sectional view of an embodiment of a system in accordance with principles described herein for reducing a microbial and/or viral load on equipment comprising a polymeric material using ozone and/or other ROSs;

FIG. 8 is a flowchart illustrating an embodiment of a method in accordance with principles described herein for reducing a microbial and/or viral load on equipment comprising a polymeric material using ozone and/or other ROSs; and

FIGS. 9-37 are graphs illustrating testing data pertaining to different embodiments of systems and methods for reducing a microbial and/or viral load on equipment comprising a polymeric material using ozone and/or other ROSs.

### DETAILED DESCRIPTION OF THE DISCLOSED EMBODIMENTS

The following discussion is directed to various exemplary embodiments. However, one skilled in the art will understand that the examples disclosed herein have broad application, and that the discussion of any embodiment is meant only to be exemplary of that embodiment, and not intended to suggest that the scope of the disclosure, including the claims, is limited to that embodiment.

Certain terms are used throughout the following description and claims to refer to particular features or components. As one skilled in the art will appreciate, different persons may refer to the same feature or component by different names. This document does not intend to distinguish between components or features that differ in name but not function. The drawing figures are not necessarily to scale. Certain features and components herein may be shown exaggerated in scale or in somewhat schematic form and some details of conventional elements may not be shown in interest of clarity and conciseness.

In the following discussion and in the claims, the terms “including” and “comprising” are used in an open-ended fashion, and thus should be interpreted to mean “including, but not limited to . . . ” Also, the term “couple” or “couples” is intended to mean either an indirect or direct connection. Thus, if a first device couples to a second device, that connection may be through a direct connection, or through an indirect connection via other devices, components, and connections. In addition, as used herein, the terms “axial” and “axially” generally mean along or parallel to a central axis (e.g., central axis of a body or a port), while the terms “radial” and “radially” generally mean perpendicular to the central axis. For instance, an axial distance refers to a distance measured along or parallel to the central axis, and a radial distance means a distance measured perpendicular to the central axis.

As described above, reactive species, including ROSs, may be generated as a plasma from a suitable plasma generator such as a DBD reactor. Particularly, ozone is a ROS that is an effective oxidizer capable of effectively killing microorganisms through an inactivation process. Ozone may be generated and delivered as a plasma to the surfaces of the equipment to be treated using a plasma generator such as a DBD reactor. Relative to other treating agents, ozone may offer some advantages in at least some applications due to ozone's antiviral profile, relatively short half-life, and gaseous and diffusive nature.

The biocidal and viricidal capabilities of ozone may depend on several factors. Not intending to be bound by any particular theory, a microorganism survival faction (SF) may be expressed in accordance with Equation (1) below, where “N_{S}” represents the concentration of surface viruses survived after exposure to ozone, which may be expressed in units of plaque forming units per milliliter (PFUs/mL); “N₀” represents the concentration of surface viruses before exposure to ozone, which may be expressed in units of PFUs/mL; “C” represents ozone concentration which may be expressed in units of parts per million (ppm); “t” represents ozone contact time, which may be represented in units of minute (min); and “K” represents the virus susceptibility factor, which may be represented in units of 1/(ppm-min):

$$S F = \frac{{N}_{S}}{{N}_{0}} = {e}^{- K C t} \qquad ( 1 )$$

Ozone is particularly effective against specific diseases such as, for example, Hepatitis A, Enteroviruses, rotaviruses, influenza viruses, enteric viruses, and rhinoviruses. Ozone is also particularly effective against coronaviruses due to the abundant cysteine in the spike proteins of coronaviruses. For example, a zero level of infectivity may be obtained for Theiler's murine encephalomyelitis virus (TMEV) (a coronavirus) within one to three hours if treated with approximately 200 ppm of ozone at 80% relative humidity (RH). As another example, exposures for less than an hour of 10 ppm to 20 ppm ozone at high RV can reduce viral concentrations by 99.9%. Ozone may thus, in at least some applications, disinfect equipment relatively more rapidly than other disinfecting or treatment agents.

While ROSs including ozone are effective biocides and viricides, ROSs including ozone may damage or destroy some materials when exposed to too great a dose of ozone, where the ozone dosage may be defined or quantified as the product of the contact time (t) and the ozone concentration (C) (e.g., ozone concentration in ppm) on the microorganism.

Equipment comprising polymers are particularly susceptible to damage from ROSs including ozone when exposed to relatively high doses of ROSs. Given the fragility of polymer comprising equipment to ROS and ozone exposure, systems utilizing ROSs including ozone for treating equipment have conventionally been limited to equipment that does not include polymers or other materials susceptible to damage in response to ROS ozone overexposure. These limitations have limited the viability of ROSs as agents for disinfecting equipment comprising polymers and other plastics.

Additionally, many forms of personal protective equipment (PPE) including respirators, face shields, personal protection gowns, masks, gloves, etc., comprise polymers and other materials that are susceptible to being damaged when exposed to ozone and other ROSs, thereby limiting or preventing the use of such agents in treating PPE. Accordingly, PPE may be disposed of following exposure to harmful microorganisms and/or viruses, or treated by disinfecting agents that may not have the same biocidal and viricidal properties as ROSs but do not damage the PPE during treatment. The inability to leverage the biocidal and viricidal properties of ozone and other ROSs in treating PPE may reduce the availability of PPE to healthcare workers and other personnel, potentially leading to shortages of PPE. The inability to use ozone and other ROSs in treating PPE may also increase the time, cost, and complexity associated with disinfecting or otherwise treating PPE.

Accordingly, embodiments of systems and methods for treating PPE and other equipment comprising polymers and other materials using ROSs such as ozone are described herein. Particularly, embodiments disclosed herein include systems and methods for reducing a microbial and/or viral load on equipment comprising a polymeric material using ozone and/or other ROSs. The systems and methods disclosed herein provide for at least a 3-log reduction in a microbial and/or viral load on the equipment. Systems and methods disclosed herein also provide for at least a 6-log reduction in a microbial and/or viral load on the equipment. The systems and methods disclosed herein may achieve a 3-log or greater reduction in the microbial and/or viral load without damaging or otherwise negatively effecting the treated equipment. This may be done by providing for sufficient intermixing of the ozone and/or other ROSs with air so as to provide a consistent concentration of the ozone in the ozone comprising airflow to the equipment. This may also be accomplished by minimizing the dose of ozone required to effect the 3-log or 6-log reduction by, for example, elevating a humidity of within an interior of a container comprising the equipment and the ozone generator generating the ozone. Systems described herein are also portable allowing the system to be transported to the equipment to be treated rather than needing to ship or otherwise transport the equipment to the system. In this manner, a rapid and effective means for disinfecting equipment, such as PPE comprising polymeric materials, may be provided.

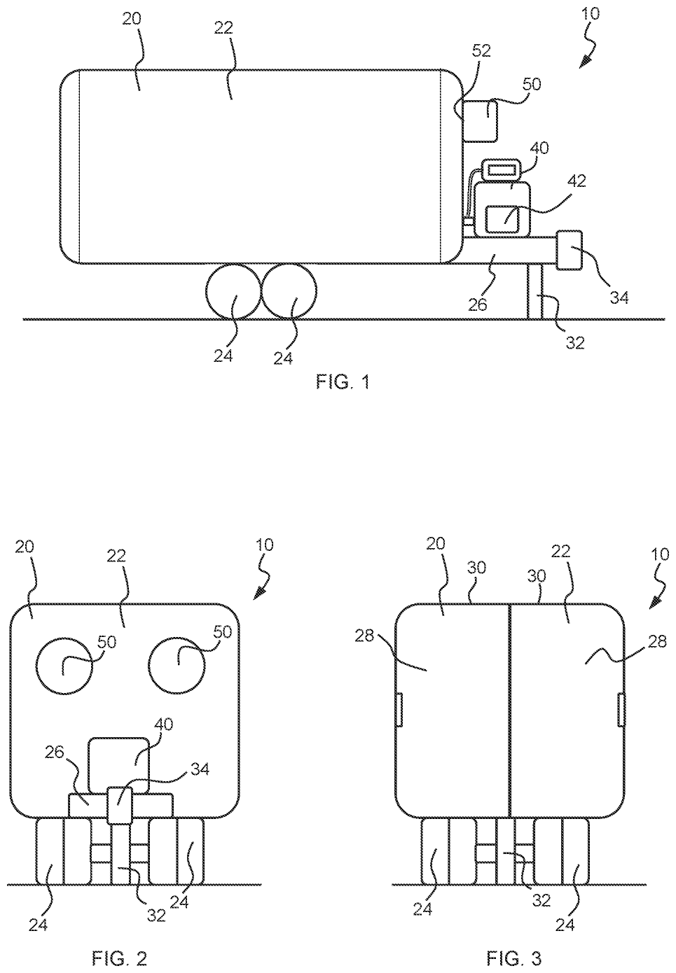

Referring to FIGS. 1-4, an embodiment of a system 10 for system for reducing a microbial and/or viral load on equipment comprising a polymeric material using ozone and/or other ROSs is shown. As will be described further herein, system 10 may be used to rapidly achieve bioburden reduction (i.e., a 3-log or 99.9% or greater reduction) of a biological and/or viral contaminant in PPE treated by system 10. Thus, system 10 may comprise a bioburden reduction system. System 10 may also be used to rapidly decontaminate (i.e., a 6-log or 99.9999% or greater reduction of the biological contaminant) PPE treated by system 10. Additionally, the PPE treatable by system 10 may comprise various polymeric materials or plastics; and may comprise porous and nonporous surfaces, soft surfaces (e.g., gowns, masks, etc.), and hard surfaces (e.g., face shields, etc.). As will be described further herein, the polymer comprising PPE may be treated by system 10 as to as to effect a 3-log or greater reduction of a biological contaminant in the PPE without damaging or otherwise rendering the PPE unsuitable for future use.

System 10 generally includes a sealable container 20 in which the PPE to be treated by system 10 may be treated. In this exemplary embodiment, container 20 comprises a road-transportable trailer, and thus, may also be referred to herein as trailer 20. Although in this embodiment container 20 comprises a trailer, in other embodiments, container 20 may comprise various kinds of sealable containers that may or not be transportable. In this embodiment, trailer 20 generally comprises a body 22 that defines an interior 23 of the trailer 20, a plurality of wheels 24, and a tongue 26 extending from a first end or front of the trailer 20. Additionally, trailer 20 comprises a pair of doors 28 positioned at a second end or rear of the body 22 of the trailer 20. Doors are openable to provide access to the interior 23 of the trailer 20. A pair of seals 30 may extend about a periphery of the doors 28 to seal the interface between the doors 28 and thereby seal the interior 23 of body 22 from an external environment surrounding the trailer 20 when the doors 28 of the trailer 20 are closed. Additionally, in this embodiment, the tongue 26 of trailer 20 includes an extendable/retractable support or post 32 and a connector 34 located at a terminal end of the tongue 26. Connector 34 may be connected to a trailer hitch of a truck or other powered vehicle so that trailer 20 may be towed to a desired location. In this manner, trailer 20 may be transported by the vehicle to the location of the PPE to be treated by the system 10.

System 10 also includes a power supply 40 that may be located external the interior 23 of trailer 20. In this embodiment, power supply 40 is conveniently supported on the tongue 26 of trailer 20; however, the location of power supply 40 may vary in other embodiments. Power supply 40 is generally configured to provide power to the powered components of system 10 as will be further described herein. In this exemplary embodiment, power supply 40 comprises a portable gas-powered generator providing approximately 10 kilowatts (kW) to 50 kW of power; however, in other embodiments, the configuration of power supply 40 may vary. For instance, the configuration of power supply 40 may vary depending upon configuration and size of the container 20. For example, the components housed within an embodiment comprising a relatively small, human-portable container 20 may be powered by a battery pack or the like. Conversely, a container 20 in the form of a room of a building or other fixed structure may be powered directly by the electrical grid servicing the fixed structure.

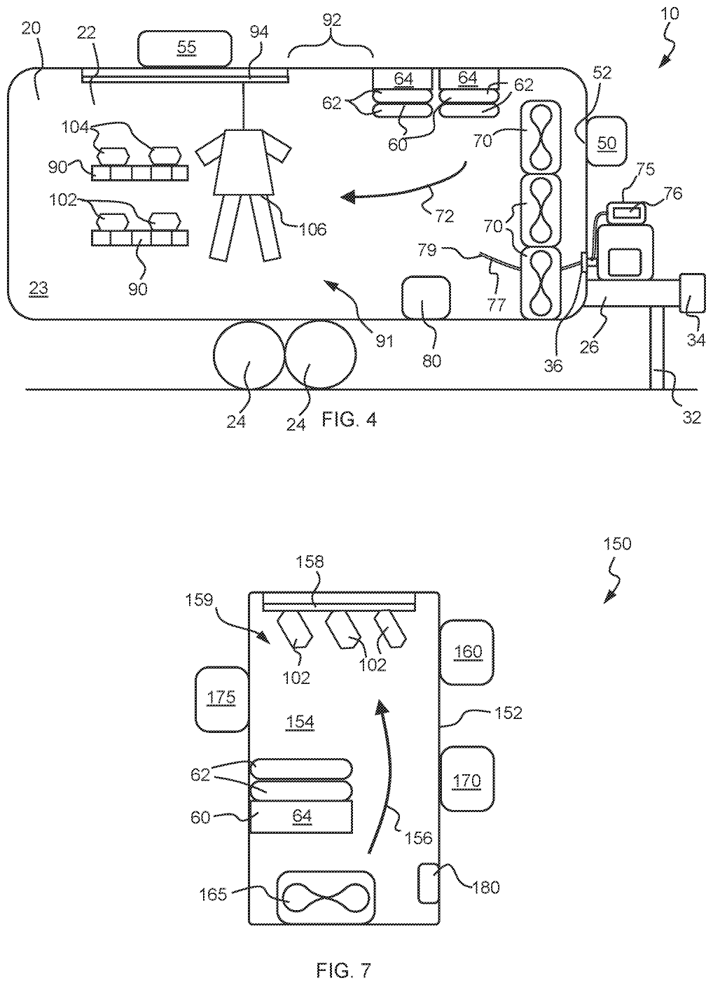

In this exemplary embodiment, power supply 40 comprises a control panel 42 from which power supply 40 may be controlled, and which may also define an interface to which the electrically powered components of system 10 may connect to form an electrical connection therewith. In this exemplary embodiment, electrical power cables may extend from components of system 10 positioned within the interior 23 of trailer 20 to control panel 42 via a sealed opening or passage 36 (shown in FIG. 4) formed in body 22. While in this embodiment power supply 40 and control panel 42 are positioned exterior of body 22, in other embodiments, power supply 40 and control panel 42 may be located within the interior 23 of body 22, such as within an interior compartment that is sealed from a remainder of the interior 23 of body 22.

Additionally, one or more exhaust fans 50 are supported on the exterior of the body 22. Exhaust fans 50 are configured to vent the interior 23 of trailer 20 following one or more treatment cycles of PPE located within interior 23. In this exemplary embodiment, a valve 52 is positioned at the interface formed between each exhaust fan 50 and the interior 23 of trailer 20. Valves 52 may be closed during the performance of a treatment cycle of PPE to ensure fluidic isolation between the interior 23 of trailer 20 and the external environment during the treatment cycle. Valves 52 may be opened following the performance of the treatment cycle to allow for venting of the interior 23 of trailer 20. Exhaust fans 50 may thus vent ozone from the interior 23 of trailer 20 to a level that does not present a danger to personnel that may enter the interior 23 of trailer 20 following the performance of the treatment cycle.

In this exemplary embodiment, system 10 also includes an air conditioning (A/C) unit 55 supported on the body 22 of trailer 20. NC unit 55 is generally configured to maintain the interior 23 of trailer 20 at a desired temperature during the duration of a treatment cycle of system 10. A/C unit 55 may also maintain fluidic isolation between the interior 23 of trailer 20 and the external environment as A/C unit 55 maintains the desired temperature within interior 23. In some embodiments, NC unit 55 may comprise a heat pump or other mechanism that allows for the control of temperature within interior 23 while maintaining a seal between interior 23 and the external environment. In other embodiments, system 10 may not include NC unit 55. For instance, in embodiments in which trailer 20 comprises instead a human-portable container, the container may be positioned within air-conditioned room or other area set at the desired temperature. In still other embodiments, the temperature within interior 23 may not be controlled but instead may be allowed to vary within acceptable limits. In some embodiments, A/C unit 55 may comprise a humidity sensor for monitoring humidity within interior 23 of trailer 20 and/or a temperature sensor for monitoring the temperature within interior 23. In other embodiments, a separate sensor package may be utilized to monitor humidity and/or temperature within the interior 23 of trailer 20.

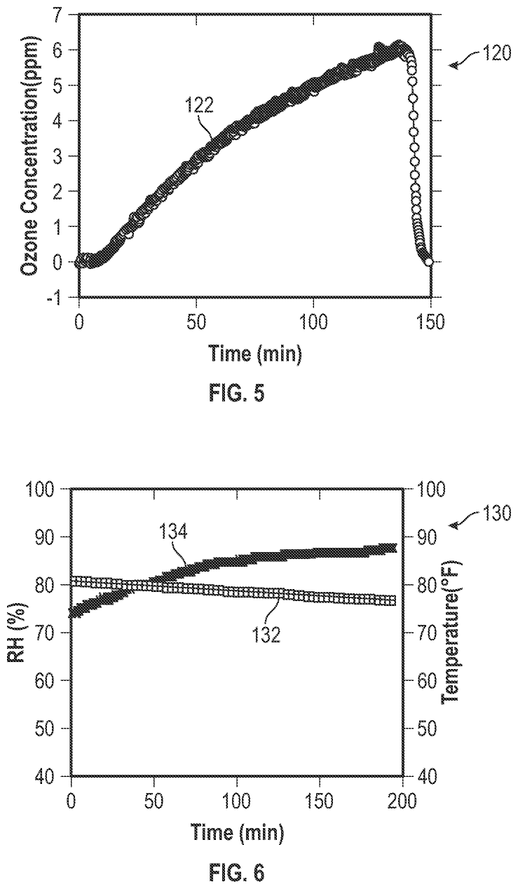

As best shown in FIGS. 4 and 5, in this exemplary embodiment, system 10 also includes a plurality of ozone generators 60, a plurality of circulation fans 70, an ozone detector 75, and a humidifier 80. Ozone generators 60 are each electrically connected to power supply 40 and are generally configured to generate and discharge an ozone and/or other ROSs using electrical energy supplied by power supply 40. Ozone generators 60 are positioned within the interior 23 of trailer 20. Particularly, in this exemplary embodiment, ozone generators 60 are each suspended from a ceiling of the interior 23 of trailer 20; however, in other embodiments, the location and manner of supporting ozone generators 60 within trailer 20 may vary.

In this exemplary embodiment, each ozone generator 60 comprises a plasma generator such as a DBD reactor generally including a circuit box 62 and a plurality of DBD tubes or electrodes 64 electrically connected to the circuit box 62. The circuit box 62 of each ozone generator 60 receives an input voltage from power supply and increases the received input voltage via a high-voltage transformer to a voltage sufficient for DBD plasma generation with electrodes 64. For example, in some embodiments, the circuit box 62 may convert a 110 volt (V) input voltage in an approximately 1.0 kilovolt (kV) to 4.0 kV output voltage at an approximately 60 hertz (Hz) frequency.

Each electrode 64 generally includes an inner cylindrical perforated electrode, an intermediate dielectric barrier in the form of a quartz tube, and an outer meshed electrode. The high output voltage provided by the circuit box 62 to the electrode 64 ignites two types of plasma—filamentary and surface DBD—due to the gap distances between the outer meshed electrode and the intermediate dielectric barrier. In this exemplary embodiment, gaseous ozone is generated from the plasma generated by each ozone generator 60 from a three-body reaction involving O and O2, leading to the formation of O3 molecules. Free radicals, such as, for example, OH*, HO2*, O2—, H3O+, N2+, and radicals NO, NO2, H2O2, and O2 (1Δg) may also be formed from gaseous O3 generated by the ozone generators 60. In some embodiments, ozone generators 60 may comprise model 5350 and/or model 5550 DBD reactors provided by Aerisa, Inc. However, in other embodiments, ozone generators 60 may comprise other types of plasma generators besides DBD reactors such as, for example, needle plasma emitters. In some embodiments, each of the ozone generators 60 generates approximately between 0.10 liters per hour (L/hr) and 0.50 L/hr of ozone; however, the rate of ozone generation of plasma generators may vary.

In this exemplary embodiment, the amount of ozone generated by each ozone generator 60 may be manually adjusted by an operator of system 10 by adjusting the amount of output voltage supplied to electrodes 64 from the circuit box 62. The operation of ozone generators 60 may thus be pre-set by an operator of system 10 prior to the performance of a treatment cycle. In other embodiments, the operation of ozone generators 60 may be controlled at control panel 42 or another location permitting the operation of ozone generators 60 to be controlled during the performance of a treatment cycle.

The circulation fans 70 of system 10 are positioned proximal the front of trailer 20 within the interior 23 thereof. Although in this embodiment system 10 includes a plurality of circulation fans 70, in other embodiments, system 10 may include a single circulation fan 70 or other device configured to induce an airflow within the interior 23 of trailer 20. Circulation fans 70 are electrically powered by power supply 40 in this embodiment, but may be powered by other sources (e.g., batteries, etc.) in other embodiments. Circulation fans 70 are generally configured to intermix the ozone generated by ozone generators 60 with the ambient air within the interior 23 of trailer 20 (the interior 23 of trailer 20 being sealed from the external environment during the operation of fans 70). Particularly, circulation fans 70 are configured to establish a flowpath 72 of ozone containing air at a desired flowrate. In some embodiments, the flowrate established by circulation fans 70 of the ozone containing air along flowpath 72 is approximately between 0.5 feet per minute (ft/min) and 10.0 ft/min; however, in other embodiments, the flowrate provided by circulation fans 70 may vary.

The ozone detector 75 of system 10 is configured to detect or determine the ozone concentration (e.g., concentration in ppm) of ozone in the airflow along flowpath 72 provided by circulation fans 70. Particularly, ozone detector 75 is positioned exterior of trailer 20 and is electrically powered by power supply 40 in this embodiment, but may be powered by other sources (e.g., batteries, etc.) in other embodiments. Ozone detector 75 may comprise a model 106 series ozone monitor provided by 2b Technologies; however, a variety of ozone detectors or monitors may be used in system 10. A fluid conduit or tube 77 extends from ozone detector 75 and through the opening 36 formed in trailer 20 such that a terminal end or opening 79 of tube 77 is located at a desired position within the interior 23 of trailer 20. For example, the opening 79 of tube 77 may be positioned at a predefined height above a floor of the trailer 20 and within the vicinity of the flowpath 72.

In this exemplary embodiment, ozone detector 75 includes a display 76 from which current measurements of the ozone concentration within the interior 23 of trailer 20 may be monitored. In other embodiments ozone concentration measurements provided by ozone detector 75 may be transmitted to other devices (e.g., a smartphone of the operator of system 10) where they may be monitored. Ozone detector 75 may be used to confirm that a predefined ozone concentration in the airflow provided along flowpath 72 falls within a predefined range corresponding to a predefined, desired ozone dosage to be delivered during a given treatment cycle. Ozone detector 75 may also inform an operator of system 10 that exhaust fans 50 have successfully reduced the ozone concentration within the interior 23 of trailer 20 to a tolerable and safe level (e.g., less than 0.1 ppm) following the performance of the treatment cycle so that personnel may safely open and enter the interior 23 of trailer 20.

Humidifier 80 of system 10 is also positioned within the interior 23 of trailer 20 and is generally configured to provide a desired RH within the interior 23 of trailer 20. Particularly, humidifier 80 is configured to maintain a relatively high RH within the interior 23 of trailer 20 during the performance of a treatment cycle to enhance the biocidal and viricidal properties of the ozone generated by ozone generators 60. In some embodiments, humidifier 80 maintains the interior 23 of trailer 20 at a humidity of approximately between 75% RH and 95% RH during the entirety of a given treatment cycle. In some embodiments, humidifier 80 maintains the interior 23 of trailer 20 at a humidity of approximately between 75% RH and 85% RH during the entirety of a given treatment cycle.

As described above elevating the humidity within the interior 23 of trailer 20 may enhance the biocidal and viricidal properties of the ozone generated by ozone generators 60. Particularly, not intending to be bound to any particular theory, the virus susceptibility factor (K factor) is largest with respect to ozone exposure for humidity in the range of 70% RH to 90% RH. For example, high humidity may lead to an increase in the generation of peroxide species thereby resulting in an increase in the K factor. Additionally, microbes to be treated are in a wetted state at higher humidity allowing for easier absorption of disinfecting agents and easier transport around microbes and through cellular walls. In view of the positive correlation between RH and the virus susceptibility factor, by elevating the humidity within the interior 23 of trailer 20 using humidifier 80, a lower dosage of ozone may be utilized during a given treatment cycle to effectively treat PPE. The reduced dosage of ozone may in-turn prevent or at least mitigate any effect of the ozone dosage on the PPE as will be discussed further herein.

In this exemplary embodiment, system 10 additionally includes a plurality of wire racks or shelves 90 are positioned within the interior 23 of trailer 20. Wire shelves 90 may be used to ensure airflow may pass evenly through the shelves 90 such that shelves 90 do not impede or obstruct airflow along flowpath 72. In some embodiments, wire shelves 90 comprise metal wire racks coated with a polyurethane plastic coating; however, in other embodiments, the materials comprising wire shelves 90 may vary. Wire shelves 90 may be attached to an interior wall of the trailer 20. In addition, a cylindrical coat rack 94 may be suspended from the ceiling of trailer 22.

In this embodiment, shelves 90 and rack 94 are located in a receptive region 91 of the interior 23 of trailer 20 configured to receive equipment to be treated by system 10. Flowpath 72 provided by circulation fans 70 may extend unobstructed towards and into the receptive region 91. Receptive region 91 is disposed at a predefined distance 92 from the plurality of ozone generators 60 of system 10. In some embodiments, predefined distance 92 may be between approximately 0.05 feet (ft) to 3.0 ft depending on the flowrate delivered by circulation fans 70. Particularly, the distance 92 and flowrate provided by circulation fans 70 define a diffusion time period during which ozone generated by ozone generators 60 may diffuse prior to contacting equipment supported by wire shelves 90 and rack 94. Thus, an increase in distance 92 may be offset by an increase in the flowrate provided by circulation fans 70 to maintain a desired diffusion time sufficient to adequately mix ozone with the air circulated in the airflow flowing along flowpath 72.

In some embodiments, the diffusion time may be between five second and 90 seconds whereby at least one of an at least 3-log reduction in a microbial and/or viral load may be reduced in response to an exposure of an ozone dose between 450 ppm-min and 550 ppm-min, and an at least 6-log reduction in the microbial and/or viral load may be reduced in response to an exposure of an ozone dose between 1450 ppm-min and 1550 ppm-min. In some embodiments, a ratio between the predefined distance 92 and the flowrate provided by circulation fans 70 may be between five and 90. In some embodiments, a ratio between the predefined distance 92 and the flowrate provided by circulation fans 70 may be between 20 and 45. These ratios provide for a diffusion time sufficient to effect the 3-log and 6-log reductions described above in response to the 450 ppm-min to 550 ppm-min ozone dose, and the 1450 ppm-min to 1550 ppm-min ozone dose, respectively. These ratios also allow for adequate diffusion of the ozone and other active species to avoid the possibility of an undesirably high concentration of poorly mixed ozone contacting and potentially damaging the equipment to be treated.

A plurality of equipment or PPE 100, 102 may be positioned on the wire shelves 90 for treating during a treatment cycle of system 10. Additionally, equipment or PPE 104 may be suspended from coat rack 94. In this exemplary embodiment, PPE 100 comprise respirators 100, PPE 102 comprise surgical masks 102, and PPE 104 comprises a surgical gown 106. Each of PPE 100, 102, 104 include polymers and/or other plastic materials that may become damaged from overexposure to ozone. The type of PPE positioned on wire shelves 90 and rack 94 of trailer 20 may vary depending on the particular application. For example, other types of PPE such as face shields, gloves, etc., may be positioned on wire shelves 90 for treating by system 10. Additionally, other types of equipment besides PPE may be positioned within trailer 20 and treated by system 10. For instance, certain medical equipment and/or other types of equipment in need of decontamination may be positioned within trailer 20 and treated by system 10.

PPE 100, 102, 104 are each received in the receptive region 91 separated from the ozone generators 60 by the predefined distance 92. The spacing of PPE 100, 102, 104 from ozone generators 60 provides for sufficient time to allow the ozone generated by ozone generators 60 to diffuse uniformly within the air forming the airflow flowing along flowpath 72. In this manner, the concentration of ozone may be substantially uniform within the airflow flowing along flowpath 72 such that the PPE 100, 102, 104 is not exposed to highly concentrated, poorly mixed and diffused ozone that could otherwise potentially damage the PPE 100, 102, 104. Additionally, no obstructions are provided between the ozone generators 60, circulation fans 70, and the PPE 100, 102, 104 (which could otherwise disturb the airflow) to further enhance uniform mixing and diffusion of the ozone within the airflow flowing along flowpath 72. By uniformly mixing the ozone within the airflow flowing along flowpath 72, only the desired concentration of ozone may be contacted with the PPE 100, 102, 104, thereby avoiding exposure of PPE 100, 102, 104 to air containing ozone at a concentration substantially greater than the desired ozone concentration.

As described above, equipment, such as PPE 100, 102, 104, may be treated by system 10 via performing a treatment cycle. Particularly, during a treatment cycle of system 10, equipment positioned within the interior 23 of trailer 20 is exposed to a predefined dose of ozone and/or other ROSs for a predefined period of time. For example, to achieve at least a 3-log reduction of microbial and/or viral load, the equipment may be exposed to a 500 ppm-min dose of ozone over the course of approximately 150 minutes. To begin this exemplary treatment cycle equipment in the form of PPE 100, 102, 104 may be positioned on wire shelves 90 and rack 94. The doors 28 of trailer 20 may then be closed to seal the interior 23 of trailer 20 from the external environment. Ozone generators 60 may be activated following the closure of doors 28 to initiate the treatment cycle. Humidity within the interior 23 of trailer 20 may be controlled by humidifier 80 and maintained between 75% RH and 95% HR during the duration of the treatment cycle.

Referring briefly to FIGS. 5, 6, graphs 120, 130, respectively, are shown. Particularly, graph 120 of FIG. 5 depicts ozone concentration 120 in units of PPM over the duration of the treatment cycle. Graph 130 of FIG. 5 depicts both humidity 132 in units of % RH and temperature in units of Fahrenheit (° F.) over the duration of the treatment cycle. As shown particularly in FIG. 5, the ozone concentration peaks at approximately 6 ppm in this example prior to the shut-off of ozone generators 60. Additionally, as shown particularly in FIG. 6, humidity is maintained between approximately 75% RH and 85% RH while temperature is maintained approximately between 82° F. and 76° F. during the duration of the treatment cycle.

Referring again to FIGS. 1-4, in this example, once the 500 ppm-min dose of ozone has been delivered to PPE 100, 102, 104, ozone generators 60 may be deactivated to cease the generation of ozone within the interior 23 of trailer 20. Valves 52 may then be opened and exhaust fans 50 may be activated to vent the ozone within the interior 23 of trailer 20 to a concentration to a safe level. Once a safe level of ozone has been established within the interior 23 of trailer 20, as indicated by ozone detector 75, the treated PPE 100, 102, 104 may be retrieved from trailer 20. Thus, a 3-log or greater microbial and/or viral load reduction may be achieved with respect to PPE 100, 102, 104 in the span of only a few hours using system 10. Additionally, due to the mobility of system 10, the PPE 100, 102 and 104 to be treated need not be transported or shipped and instead system 10 may be transported to the location of PPE 100, 102, 104 where the PPE 100, 102, 104 may be rapidly treated by the system 10.

In some embodiment, it may be desirable to effect a greater microbial and/or viral load reduction greater than 3-log. For instance, it may be desired to effect a 6-log or greater microbial and/or viral load reduction in PPE 100, 102, 104. In some embodiments, a 6-log or greater microbial and/or viral load reduction in PPE 100, 102, 104 (or other equipment) may be achieved by exposing PPE 100, 102, 104 to an ozone dose of approximately 1500 ppm-min. A 1500 ppm-min dose of ozone may be administered to PPE 100, 102 and 104 by repeating the 500 ppm-min treatment cycle above sequentially three times. Alternatively, the time period of the 500 ppm-min treatment cycle may be tripled while maintaining a similar ozone concentration. As a further alternative, in instances where the equipment to be treated has a relatively higher tolerance to ozone exposure, the rate of ozone generation produced by ozone generators 60 may be increased to minimize the time required to effect a 6-log or greater reduction in microbial and/or viral load.

Referring to FIG. 7, another embodiment of a system 150 for system for reducing a microbial and/or viral load on equipment comprising a polymeric material using ozone and/or other ROSs is shown. Similar to system 10 shown in FIGS. 1-4, system 150 used to both rapidly achieve bioburden reduction (i.e. a 3-log or 99.9% or greater reduction) of a biological or viral contaminant in equipment (e.g., PPE), and to rapidly decontaminate (i.e., a 6-log or 99.9999% or greater reduction of the biological or viral contaminant) equipment treated by system 150. In this exemplary embodiment, system 150 generally comprises a container 152, an ozone generator 60, an exhaust fan 160, a circulation fan 165, an ozone detector 170, a humidifier 175, and a sensor package 180.

In this exemplary embodiment, container 152 of system 150 comprises a human-portable container or glovebox 152 that may be manually carried by one or more people. Thus, system 150 may be transported to a desired location without needing to tow system 150 along a roadway. Glovebox 152 includes a sealable door or hatch to provide access to an interior of the glovebox 152 while allowing for the interior 154 to remain sealed or fluidically isolated from the environment surrounding glovebox 152 when the door is sealed.

Ozone generator 60 and circulation fan 165 are positioned within the interior such that an unobstructed flowpath 156 of an ozone containing airflow may be provided by circulation fan 165 and that extends towards the equipment receivable within the interior 154 of glovebox 152. Circulation fan 165 may be configured similarly as the circulation fans 70 shown in FIG. 4. In this exemplary embodiment, glovebox 152 comprises a rack 158 from which one or more pieces of equipment may be suspended, such as, for example, PPE 102 shown in FIG. 5. The equipment suspended from rack 158 is positioned in a receptive region 159 of the interior 154 of glovebox 152 that is spaced from the ozone generator 60 by a predefined distance.

Similar to exhaust fan 50 shown in FIGS. 1, 2, and 4, the exhaust fan 160 of system 150 is generally configured to vent or evacuate ozone from the interior 154 of glovebox 152 following the performance of a treatment cycle. In some embodiments, exhaust fan 160 may comprise a hood configured to receive ozone and direct the ozone away from the system 150. Additionally, the humidifier 175 is similar in operation to the humidifier 80 shown in FIG. 4 and is generally configured to maintain an elevated humidity (e.g., between approximately 75% RH and 95% RH) within the interior 154 of glovebox 152. Additionally, ozone detector 170 is similar in operation to the ozone detector 75 shown in FIG. 4 and is generally configured to monitor the ozone concentration within the interior 154 of glovebox 152. Ozone detector 170 may be used to conform that a correct dosage of ozone (e.g., 500 ppm-min, 1500 ppm-min, etc.) was administered to the equipment to be treated and/or to conform that the ozone within the interior 154 of glovebox 152 was successfully vented by exhaust fan 160 following the performance of a treatment cycle such that glovebox 152 may be safely opened.

In this exemplary embodiment, system 150 does not comprise a dedicated A/C unit specific to the glovebox 152. However, given that glovebox 152 is human-portable, it may be positioned within a structure or room that is temperature controlled, thereby allowing for the temperature control of the interior 154 of glovebox 152. Sensor package 170 of system 150 is generally configured to measure the humidity and temperature within the interior 156 of glovebox 152 to ensure the humidity and/or temperature are maintained within predefined acceptable ranges during the performance of a treatment cycle using system 150.

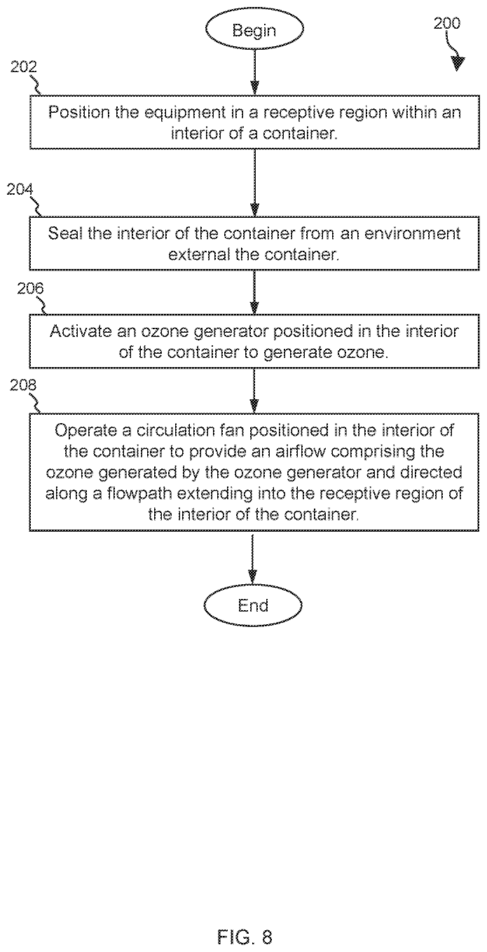

Referring to FIG. 8, an embodiment of a method 200 for reducing a microbial and/or viral load on equipment comprising a polymeric material using ozone is shown. In general, method 200 can be implemented using system 10 or system 150 previously described. Initially, block 202 of method 200 comprises positioning the equipment in a receptive region within an interior of a container. In some embodiments, block 202 comprises positioning PPE 100, 102, 104 in the receptive region 91 of the interior 23 of trailer 20 as shown in FIG. 4. In other embodiments, block 202 comprises positioning PPE 102 in the receptive region 159 of the interior 154 of glovebox 152 shown in FIG. 7.

At block 204, method 200 comprises sealing the interior of the container from an environment external the container. In some embodiments, block 204 comprises closing the doors 28 of trailer 20 to seal the interior 23 of trailer 20 from the external environment. In other embodiments, block 204 comprises closing the door of the glovebox 152 to seal the interior 154 of glovebox 152 from the external environment. At block 206, method 200 comprises activating an ozone generator positioned in the interior of the container to generate ozone. In some embodiments, block 206 comprises activating the ozone generators 60 of system 10 shown in FIG. 4 or the ozone generator 60 of system 150 shown in FIG. 7.

At block 206, method 200 comprises operating a circulation fan positioned in the interior of the container to provide an airflow comprising the ozone generated by the ozone generator and directed along a flowpath extending into the receptive region of the interior of the container. In some embodiments, block 206 comprises operating the circulation fans 70 of system 10 shown in FIG. 4 to provide airflow comprising ozone along flowpath 72 towards the receptive region 91. In other embodiments, block 206 comprises operating the circulation fan 70 of system 150 shown in FIG. 7 to provide airflow comprising ozone along flowpath 156 towards receptive region 159.

Ozone exposure experiments for material testing were performed on N95 Respirators (3M 8200, Prestige Ameritech, and BYD), KN 95 respirators (3M 9502+), gowns (AAMI and Prestige Ameritech), and raw materials of polypropylene and polyester. An initial set of baseline control samples were stored with no ozone exposure. Samples were treated with an exposure based on the dosage needed to achieve a reliable viricidal effect. Table 1 below shows different types of samples, delivered dose, and the number of total samples treated in a glovebox a trailer system which may share similarities with systems 150 and 10, respectively, described above. It should be noted that a relatively small sample size of PPE was used due to shortage of the products during the COVID19 pandemic. The respirators and masks were tested intact in both the glovebox and the trailer as discussed below. For mechanical properties testing, the gowns and raw materials were cut to size using a rotary cutter to prevent distortion in pattern lines and fraying, which is important for tensile testing and other mechanical properties testing.

TABLE 1

| | Delivered Ozone Dose (ppm-min) | | |
|---|---|---|---|
| Sample Types | Control | Glovebox | Trailer |
| AAMI Gown | 0(1), | 1800(1), 3700(1) | |
| Polypropylene | 0(3), | 700(3), 1200(3), | |
| material | | 7000(3) | |
| Polyester material | 0(3), | 700(3), 1200(3), | |
| | | 7000(3) | |
| 3M N95 (8200) | | 1600(1)*, 1800(1), | |
| | | 3300(1) | |
| 3M N95 (9502+) | | 1600(1)* | |
| BYD Respirator | 0(4)⁺, | 500(1), 1500(1), | 500(4)⁺, 1500(4)⁺ |
| | | 50000(1)* | |
| Prestige Ameritech | 0(4)⁺, | 500(1), 1500(1), | 500(4)⁺, 1500(4)⁺ |
| Respirator | | 50000(1)* | |
| Prestige Ameritech | 0(1), | 500(1), 1500(1), | 500(1), 1500(1) |
| Gown | | 50000(1)* | |

*Samples inspected at intermediate dose points.
⁺Three samples sent to CDC for testing and one for in-house testing.
Number in parenthesis after the dose value is the number of samples treated at that dose.

An Instron 5943 tensile tester with a 1-kN load cell and pneumatic side action grips was utilized for mechanical testing of the PPE materials and straps. These N95 respirators have three layers in which the inner and outer layers are made of polyester and the middle layer is the polypropylene filter, whereas others, such as the BYD respirators, have four layers including a hot air cotton layer. In addition to specimens directly taken from the N95 respirators, raw materials of polyester and polypropylene were tested, as well as AAMI and Prestige Ameritech gown specimens. The testing procedure from ASTM D 5035-11 was followed for the polyester and polypropylene filter layers, AAMI gowns, and Prestige Ameritech gowns. ASTM D412-16 and ASTM D638-14 were followed for the polyisoprene straps testing. Table 2 shown below provides details of the specimen dimensions and test speeds. Tensile tests were performed at room temperature (22° C.), and the displacement rate was fixed at 100 mm/min for the materials from the N95 respirators and the polyester and polypropylene raw materials and 300 mm/min for the AAMI and Prestige Ameritech gown specimens, following ASTM D5035 .

TABLE 2

| | Specimen | Distance Between | Displacement |
|---|---|---|---|
| Materials | Length (mm) | Grips (mm) | Rate (mm) |
| AAMI Gown | 125 | 100 | 300 |
| Polyester | 57.5 | 32.5 | 100 |
| Polypropylene | 57.5 | 32.5 | 100 |
| BYD Respirator | 57.5 | 32.5 | 100 |
| Prestige Ameritech | 57.5 | 32.5 | 100 |
| Respirator | | | |
| Prestige Ameritech | 125 | 100 | 300 |
| Gown | | | |

Color change is usually caused by external exposure and will discourage customers to utilize the products even if there is no indication of product decay. Yellowness Index (YI), defined as an indication of the degree of departure of an object color from colorless or from a preferred white toward yellow [48, 49], is used to quantify the extent of color change. This follows standardized test method ASTM E313-15, commonly used to evaluate color changes in a material caused by external exposure. The quantitative evaluation by measuring the YI was done on the N95 respirators before and after treatment. A Nikon D5600 camera was used to take pictures of the Color Checker palette and sample in the standard light environment (D65). MATLAB software was employed to determine the Yellowness Index at each specifically chosen point at the same specific region on every mask.

Surface wettability analysis of the PPE has been carried out to determine changes in performance of the PPE. Wettability of materials can be characterized by the contact angle, defined as the angle between the liquid-vapor and the solid-liquid interfaces at the point where the three phases (solid, liquid, and gas) meet [50]. Generally, the methods used for contact angle testing have been divided into static drop micro-observation and dynamic testing methods. The static drop micro-observation method was chosen for the quantitative evaluation of wettability, using distilled water droplets resting on the mask material of interest. A Nikon D5600 camera, micro-Nikon lens, and 20 mL syringe were used to image the static drop. The Low-Bond Axisymmetric Drop Shape Analysis (LBADSA) Plugin for ImageJ was employed to determine the contact angle in a given image. For each sample, six repetitions were performed.

Filtration efficiency of the respirator material depends not only upon mechanical integrity of the filter material but also on the electrostatic charge, which is applied to the material during manufacturing. Any process of disinfection may cause loss of the electrostatic charge. Literature suggests that liquids such as saline solutions, distilled water, and alcohols cause to lose the electrostatic charge in the respirator material. The polypropylene materials treated by plasma ROS were tested to measure residual charge function.

A simple setup was prepared to quantify the changes in electrostatic charge on the treated sample via measuring the lift distance of tiny glass wool fibers from various heights to the sample. The assembled setup, which comprises of a lab lift platform (holding the glass wool at a fixed height), a lab lift holding a micrometer stage, and a white board sitting on the right side of lab lift. Additionally, a setup was made to intentionally apply an electrostatic charge to the filtration material. A few fibers of glass wool (15 fibers) with 1 cm length was placed on the white board below the sample. The charge measurement was performed before charging, after charging, and on 15th day after charging to visualize the residual charge and the capability of storing charge of the polypropylene material. The sample is fixed on the micrometer stage just after the charging process and the distance between the sample and the glass wool is gradually decreased until the electrostatic attraction force on the glass wool exceeds gravity and the glass wool “jumps” the remaining distance to the sample. This jumping distance is recorded.

Both the Prestige Ameritech RP88020 and BYD DE2322 respirators were treated using a plasma ROS method in the trailer at a low ozone dose of 500 ppm-min (1 cycle) and a high ozone dose of 1500 ppm-min (3 cycles). These decontaminated respirators were sent to the National Personal Protective Technology Laboratory, Pittsburgh, for material testing. Three control, three 500 ppm-min, and three 1500 ppm-min exposed respirators were prepared. This low number of samples is due to the shortage of availability from the COVID19 pandemic. NPPTL tested both respirators using a modified version of the NIOSH Standard Test Procedure (STP) TEB-APR-STP-0059 to determine particulate filtration efficiency. The TSI, Inc. model 8130 was used at a flow rate of 85 L/min. The NPPPTL report described the test process: each respirator was tested for 10 minutes, and maximum penetration was recorded for each individual respirator using a sodium chloride aerosol with maximum concentration of 200 mg/m3.

The decontaminated respirators (Prestige Ameritech RP88020 and BYD DE2322) were sent to NPPTL, Pittsburgh for tensile strength testing of the straps. An Instron® 5943 tensile tester was used to determine changes in strap integrity. The tensile test was performed by applying the force on bottom and top straps separately. In this test, three control and six decontaminated respirators were used for study. According to the NPPTL report [55], the straps were pulled at 1 cm/s until reaching 150% strain. The samples were then held at 150% strain for 30 seconds, while the force was recorded.

For exhalation testing, the decontaminated respirators (Prestige Ameritech RP88020 and BYD DE2322) were sent to NPPTL, Pittsburgh. They used a static advanced headform (StAH) to assess the manikin fit factor of respirators. The tube extending from the bottom of the StAH is connected to an inflatable (non-latex, powder-free) bladder inside an isolated and airtight plastic cylinder. This configuration prevents any particles potentially generated by the simulator from entering the breathing zone of the StAH. A port on the cylinder is connected to a Series 1101 breathing simulator (Hans Rudolf, Inc., Shawnee, Kans.).

A simple hydrostatic pressure tester was developed for the PPE material testing. The PPE was tested using standard AATCC 127 hydrostatic pressure. The setup consisted of 8 feet long PVC tubing and 16 cm long sanitary tubing. Two PVC valves were used to control the water flow. The pressure-regulating valve was assembled in the upstream of the setup to monitor the water pressure. The sample was fixed in sanitary tubing with the help of a clamp. The water enters through a first valve, only on closing of a second valve. The surface of the gown was observed carefully as the water level continuously rises through the column. As soon as three droplets appear on the surface of the gown, valve A is closed and marked on the PVC tubing. The rise of the water stream is calculated by subtracting the sanitary tubing height from water column height.

The AATCC 42 based impact penetration standard was used to measure the resistance of fabrics by the impact of water penetration [57]. A 500 mL funnel, a high-pressure showerhead, a 45-degree angle of the test apparatus, and an iron stand were assembled. Gown material samples were prepared with a size of 150 mm and clamped at one end. A smaller size of blotting paper (0.1 gram) was inserted beneath the test sample. A 500 mL volume of distilled water in a 1000 mL beaker was poured into the funnel and allowed to spray onto the test specimen. As the spraying period was accomplished, the test specimen was carefully lifted, and the blotting paper was removed for re-weighing. At the end, the difference of two weights of the blotting paper (before and after the experiment) was observed for analysis.

Raw materials of polyester/polypropylene (associated with N95 respirators) and the AAMI gown specimens were tested with an Instron tensile tester (performed at TAMU) to determine basic mechanical properties under ozone treatment (PE/PP-Control (0 ppm-min), PE/PP-1 (700 ppm-min), PE/PP-2 (1200 ppm-min), and PE/PP-3 (7000 ppm-min)). The AAMI gown materials were treated at ozone dose of 0 ppm-min (control), 1800 ppm-min, and 3700 ppm-min. ASTM D 5035-11 (standard test method for breaking force and elongation of textile fabrics) was generally followed in terms of the testing procedure for the PPE/materials.

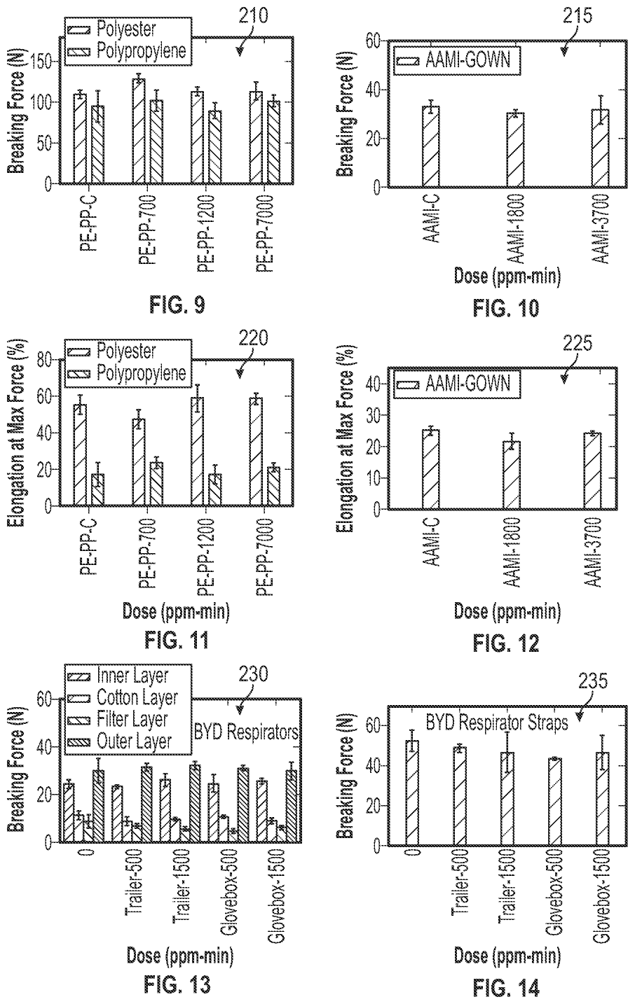

Referring to FIGS. 9-12, the breaking force of the samples (polyester and polypropylene materials, AAMI gown) from different exposure of ozone doses are shown particularly in graphs 210, 215 of FIGS. 9, 10, respectively. According to ASTM 5035 and ASTM D4848, the breaking force is defined as the maximum force exerted on the specimen, i.e., the maximum force applied to a material carried to rapture. The elongation at maximum force (%) is determined as a percentage of the length between the grips for the specimen and plotted in graphs 220, 225 of FIGS. 11, 12, respectively. The error bars represent two standard deviations from the mean. The breaking force of polyester slightly increases and the elongation at max force slightly decreases for the first dose of 700 ppm-min, however, there is no significant change of the breaking force (N) for polypropylene with ozone dose. For AAMI gowns, there is no significant change of the breaking force and elongation at maximum force with doses of Ozone.

Additionally, the ozone-treated BYD respirators were tested with an Instron tensile tester. Samples were treated at the ozone doses of 500 ppm-min and 1500 ppm-min in the glovebox, as well as 500 ppm-min and 1500 ppm-min in the trailer system. The samples for tensile testing include four nonwoven fabrics of respirator, namely inner (polypropylene spunbond), hot air cotton, filter (polypropylene melt-blown), and outer (polypropylene spunbond) layers, as well as the strap material.

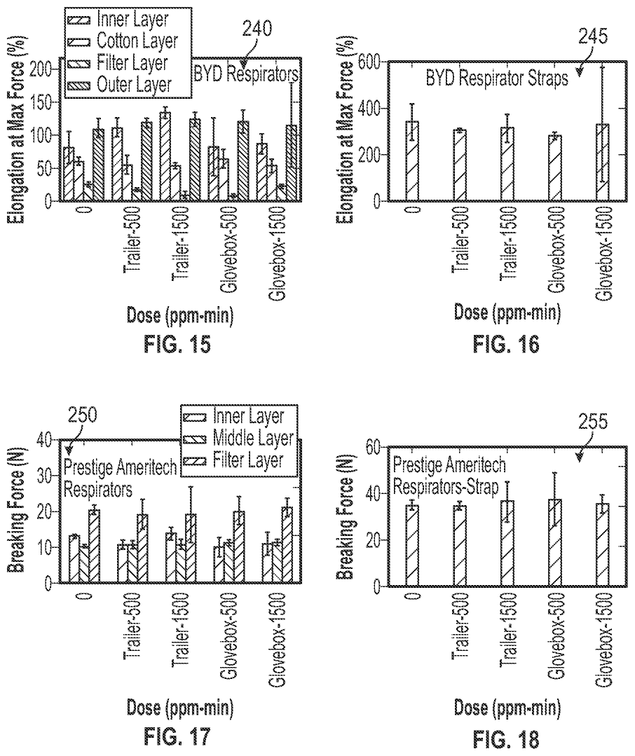

Referring to FIGS. 13, 14, the breaking force of the samples from different doses are shown in graphs 230, 235 of FIGS. 13, 14, respectively. The error bars represent two standard deviations. Referring to FIGS. 15, 16, the elongation at maximum force (%) for the specimens is presented in graphs 240, 245 of FIGS. 15, 16, respectively. There is no significant change of the breaking force and elongation at maximum force for the three layers of BYD respirators with ozone dose; however, for the filter layer, the properties decrease slightly with dose.

Similarly, the ozone-treated Prestige Ameritech (PA) respirators, straps, and gowns were tested with an Instron tensile tester. Samples were treated at the ozone dose of 500 ppm-min and 1500 ppm-min in the glovebox, as well as 500 ppm-min and 1500 ppm-min in the trailer system. The sample for tensile testing includes three layers of fabric (inner, middle and outer) and strap material.

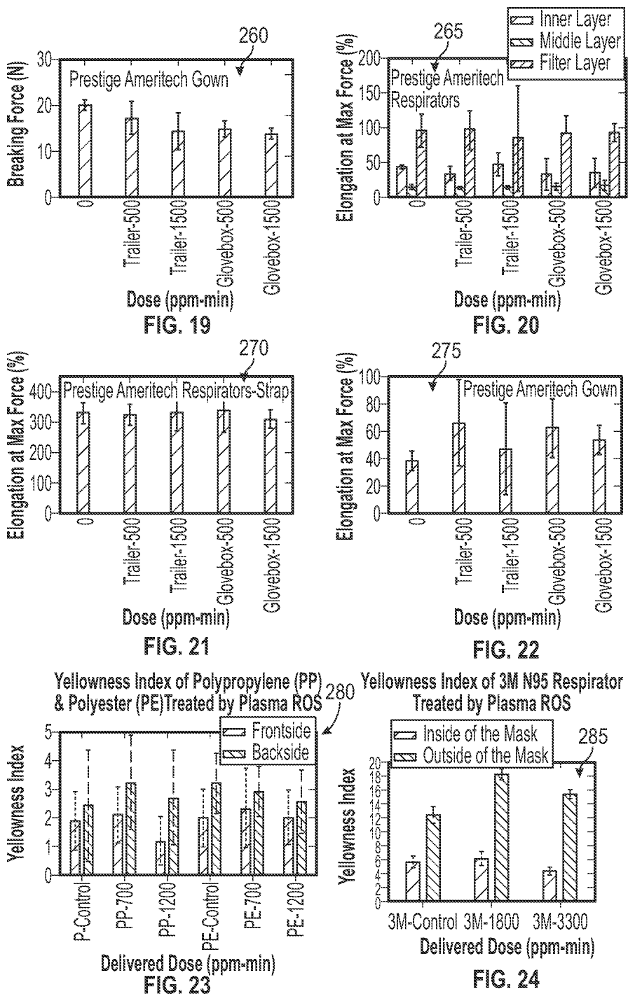

Referring to FIGS. 17-19, the breaking force of the samples from different doses are shown in graphs 250, 255, and 260 of FIGS. 17, 18, and 19, respectively, and the elongation at maximum force of the PA respirators and gown is shown in graphs 265, 270, and 275 of FIGS. 20, 21, and 22, respectively. The breaking force of the PA gown decreases slightly with dose relative to the control; otherwise, no significant changes with dose were observed.

The color change of plasma ROS treated samples was evaluated by giving the value of yellowness index (YI), a quantitative number based on X, Y, Z color space. The inside and outside of N95 respirators, front and back sides of polypropylene and polyester materials, and both sides of BYD and Prestige Ameritech respirators have been processed to analyze the YI. Referring to FIG. 23, graph 280 of FIG. 23 indicates no difference in the polypropylene (PP) and polyester (PE) materials, while the backside of PP and PE have averaged higher YI compared to front side of PP and PE. The large error bars are due to low uniformity and large sample areas used for analysis. More importantly, the yellowness index differences (ΔYI) between control sample and treated samples are all below 5, which is effectively imperceptible to the human eye.

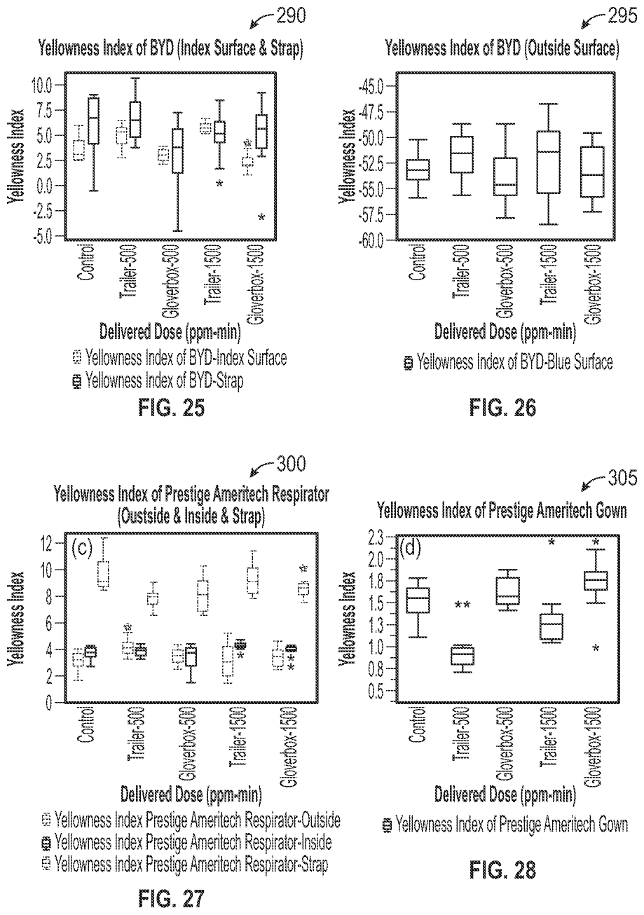

The yellowness index of the 3M N95 respirator also shows little dependence on the delivered dose. Referring to FIGS. 24-28, It is observed from the graph 285 of FIG. 24 that the outside of respirator has higher YI compared to the inside surface. Additionally, the graphs 290, 295, 300, and 305 of FIGS. 25, 26, 27, and 28 presents the YI values of BYD, Prestige Ameritech respirators, straps, and gown, respectively. There are no visually observable differences in YI in terms of the different delivered dose (control, 500 ppm-min and 1500 ppm-min). A change in YI of about 5 is needed to be noticeable to humans.

A self-assembled hydrostatic pressure tester was equipped to measure the hydrostatic pressure of AAMI and Prestige Ameritech gowns treated by plasma ROS. Table 3 below shows the values of the hydrostatic pressure of AAMI and Prestige Ameritech gowns, which were treated at two different ozone doses. Note that the maximum pressure able to be recorded with this system is 3.194 psi, which was exceeded by many samples. A moderate water resistant gown should have hydrostatic pressure higher than 0.71 psi according to the standard reported by CDC. Only the AAMI gown with a delivered dose of 3722 ppm-min shows poor results of the testing, being below the 0.71 psi pressure threshold. All trailer treated samples (both 1 and 3 cycles of the Prestige Ameritech Gown) pass this test.

TABLE 3

| | | | | | PA- | PA- | PA- | PA- |
|---|---|---|---|---|---|---|---|---|
| | | AAMI- | AAMI- | | Trailer- | Glovebox- | Trailer- | Glovebox- |
| | | 1800 | 3700 | | 500 | 500 | 1500 | 1500 |
| | | ppm- | ppm- | | ppm- | ppm- | ppm- | ppm- |
| Sample | AAMI-C | min | min | PA-C | min | min | min | min |
| Repetition 1 | 1.191 | 1.265 | 0.451 | >3.194 | 3.086 | >3.194 | 3.194 | >3.194 |
| Repetition 1 | 1.267 | 1.164 | 0.684 | >3.194 | 3.194 | >3.194 | >3.194 | 3.045 |
| Repetition 1 | 1.182 | 1.135 | 0.721 | >3.194 | >3.194 | >3.194 | >3.194 | 2.901 |
| Average | 1.213 | 1.188 | 0.618 | >3.194 | >3.16 | >3.158 | >3.194 | >3.047 |
| STDEV | 0.038 | 0.056 | 0.119 | N/A | N/A | N/A | N/A | N/A |

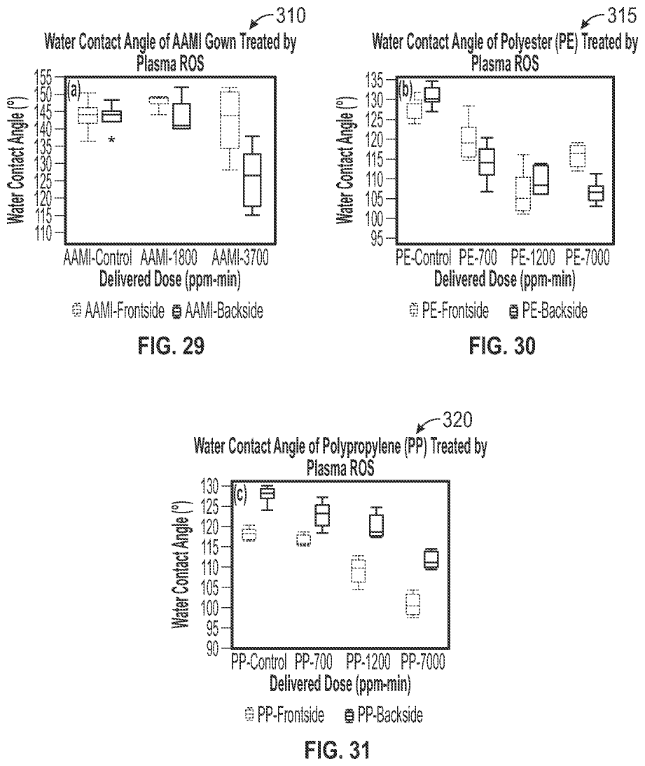

Referring to FIGS. 29-31, graphs 310, 315, and 320 of FIGS. 29-31 illustrate water contact angle for different materials. Particularly, graph 310 of FIG. 29 shows that the frontside of AAMI Gown has negligible differences with the control average value. However, the backside of the AAMI Gown indicates a large decrease of the AAMI-3700 compared with AAMI-Control and AAMI-1800. The results of both polypropylene (PP) and polyester (PE) indicate a continuous decrease of water contact angle with an increase of delivered dose occurring both at the frontside and backside. However, the situation of the frontside of PE-7000 does not follow this rule. In summary, plasma ROS treatment introduces relatively negative effects on the hydrophobic property, but they still maintain general hydrophobicity with a contact angle greater than 90°.

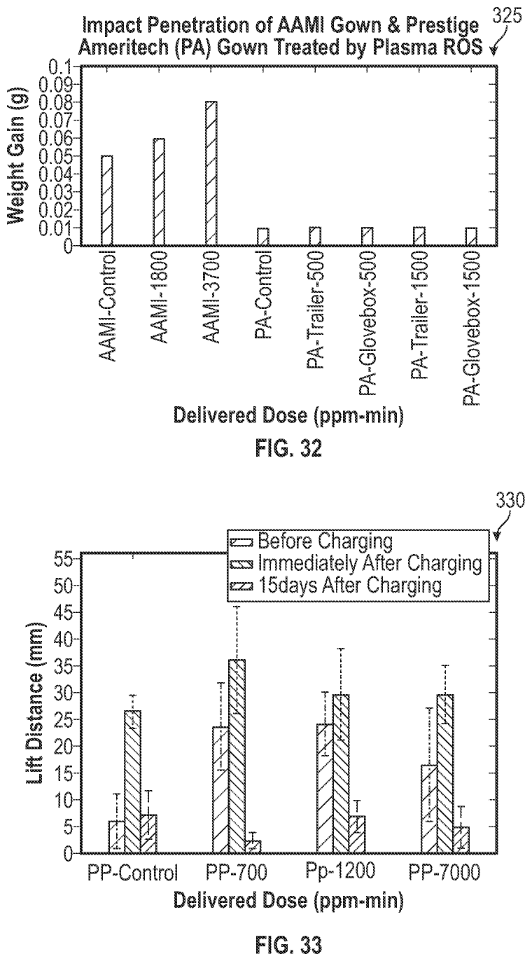

A simple method was implemented for impact penetration testing based on the AATCC test method 42-2017. According to this standard, the testing guidelines of moderate water resistance gowns should be satisfied with the AAMI and PA gowns. It is noted from the standard that the weight gain of blotting paper should be less than 1 gram. For the limited samples treated, the AAMI and PA gowns passed the requirement. Referring to FIG. 32, graph 325 of FIG. 32 shows the variation of weight gain of blotting papers for differently treated AAMI and PA gowns.

Surface charge was measured for polypropylene (PP) control samples and treated samples exposed to 700 ppm-min, 1200 ppm-min, and 7000 ppm-min dose levels. As discussed previously, the corona array setup is used to quantify the changes in electrostatic charge on the treated sample in terms of the ‘lift distance’ parameter. Referring to FIG. 33, graph 330 of FIG. 33 shows the lift distance of polypropylene material from before charging, immediately after charging, and 15 days after charging. An average value from six repetitive experiments was employed for surface charge analysis.

It is observed that the lift distance measured from plasma ROS treated PP samples show relatively higher values compared with the control samples. Plasma ROS treatment put charges on the samples. The lift distances measured immediately after charging and measured 15 days after charging indicate that the plasma ROS treatment did not significantly change the ability of the material to hold charge. The charges applied during the plasma ROS treatment of respirators did not alter the respirator filter efficiency as discussed below.

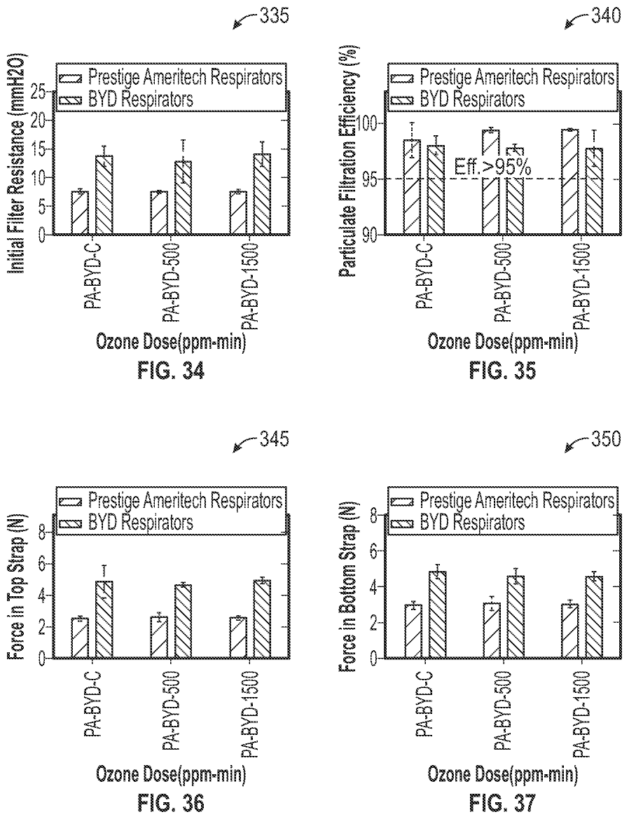

An assessment was developed to quantify the filtration efficiency and manikin fit factor of N95 respirators (Prestige Ameritech RP 88020 and BYD DE 2322) by the NPPTL group. A number of three control of each type respirators (PA-C, BYD-C), 500 ppm-min exposed samples (PA-1cycle, BYD-1 cycle), and 1500 ppm-min exposed samples (PA-3 cycle, BYD-3 cycle) were utilized for the analysis. Referring to FIGS. 34, 35, graph 335 of FIG. 34 shows a bar chart of the initial filter resistance of respirators based on treatment dose values. The error bars represent two standard deviations. It is observed from the graph that there is no significant difference of initial filter resistance due to plasma ROS treatment. In addition, the values of initial filter resistance of the BYD type respirator are higher compared to Prestige Ameritech's resistance values. Similarly, graph 340 of FIG. 35 shows particulate filter efficiencies for the control samples (PA-C, BYD-C), samples treated by 500 ppm-min exposure (PA-1cycle, BYD-1 cycle), and samples treated by 1500 ppm-min exposure (PA-3 cycle, BYD-3cycle). It is observed from the graph that there is no significant variation in filtration efficiency due to increment in ozone dose level. Ranges of filter efficiency of 99.35-99.56%, 99.50-99.59%, 97.79-98.04% and 97.10-98.69% were observed for the PA-1cycle, PA-3 cycle, BYD-1 cycle, and BYD-3cycle samples, respectively. The overall particulate filter efficiencies of all treated respirators exhibit greater than 95% efficiency.

Strap integrity testing (Instron 5943 tensile tester) was performed by the NPPTL group. Tensile force in the top and bottom straps of respirators (treated by plasma ROS) was recorded at 150% strain. Referring to FIGS. 36, 37, graph 345 of FIG. 36 shows values of tensile force for the control respirators (PA-C, BYDC), 500 ppm-min exposed samples (PA-1cycle, BYD-1 cycle), and 1500 ppm-min exposed samples (PA-3 cycle, BYD-3cycle). There is not much difference in tensile force due to plasma ROS exposure in the top straps of the respirators. Similarly, graph 350 of FIG. 37 shows almost equal tensile force observed in the bottom straps of the control and treated samples. The CDC reported no visual degradation of the straps after the plasma ROS exposure. The Prestige Ameritech respirator straps and the BYD straps show no significant change in recorded force at 1 and 3 cycles.

An in-house test was performed to evaluate the integrity of straps of different type of respirators. Two respirators—a 3M 9502+ and a 3M 8200—were subjected to ozone exposure of 1600 ppm-min at an ozone concentration of approximately 20 ppm. During treatment of the 3M 8200 respirator, the straps of the respirator broke off at around 1000 ppm-min. They started wearing off at around 400 ppm-min. The 9502+ respirator was intact after treatment to 1600 ppm-min ozone exposure. However, the 3M 8200 respirator failed at the location of metallic staples where the straps are attached to respirator.

To understand the failure behavior of 3M 8200 respirator straps, polyisoprene (strap material of 3M 8200 respirators) samples were exposed to ozone inside the glove box at different dose levels. In the first setup the straps were arranged in the glovebox flat without introducing any physical stresses. In a second setup straps were induced to bend over the support causing a stress at one point. In addition, a piece of copper tape was placed along the strap to inspect whether charge deposition on the metallic staples is causing the failure at that particular location in the 3M 8200 respirator. After each cycle of ozonation process, the straps were cyclically stretched to twice their initial length ten times each to determine whether their mechanical properties had changed during treatment. In a third setup pre-stretched samples (stretched to double their length) were placed inside the glovebox treated by ozone.

After ozone exposure, the setup without induced physical stress did not show any physical damage even after 1600 ppm-min. However, straps from the setup with the specimens bent over the support started showing physical damage around 400 ppm-min near the bend region. No damage was observed near the copper tape. At around 1000 ppm-min the wear at the bend propagated throughout the width, and the straps broke into two pieces. In the final setup with stretched straps, physical damage was first observed at the point where there was a twist on the strap material. These results indicate that it is the concentrated physical stress that leads to damage. In the case of the 3 M 8200 respirator, concentrated stress is induced in strap material by the metal staple, which ultimately leads to damage.

Additionally, both Prestige Ameritech and BYD respirators were treated by ozone dose up to 50000 ppm-min (6-7 decontamination cycles). But, there is no failure of straps observed during the experiments when achieving very a high ozone dose (50000 ppm-min). The straps of both respirators were then cyclically stretched to twice their initial length ten times. However, no physical damage was observed after plasma ROS treatment.

While embodiments of the disclosure have been shown and described, modifications thereof can be made by one skilled in the art without departing from the scope or teachings herein. The embodiments described herein are exemplary only and are not limiting. Many variations and modifications of the systems, apparatus, and processes described herein are possible and are within the scope of the disclosure. For example, the relative dimensions of various parts, the materials from which the various parts are made, and other parameters can be varied. Accordingly, the scope of protection is not limited to the embodiments described herein, but is only limited by the claims that follow, the scope of which shall include all equivalents of the subject matter of the claims. Unless expressly stated otherwise, the steps in a method claim may be performed in any order. The recitation of identifiers such as (a), (b), (c) or (1), (2), (3) before steps in a method claim are not intended to and do not specify a particular order to the steps, but rather are used to simplify subsequent reference to such steps.

What is claimed is:

1. A system for reducing a microbial and/or viral load on equipment using ozone, the system comprising:
a container comprising having an open configuration to provide access to an interior of the container and a closed configuration to seal the interior from an environment external the container, and wherein the interior comprises a receptive region to receive the equipment;
a circulation fan positioned in the interior of the container; and
one or more ozone generators positioned in the interior of the container and configured to generate ozone upon activation;
wherein the circulation fan is configured to provide an airflow comprising ozone generated by the one or more ozone generators and directed along a flowpath extending into the receptive region of the interior of the container.

2. The system of claim 1, wherein the container comprises a road-transportable trailer comprising a plurality of wheels.

3. The system of claim 1, further comprising an electrical generator supported on the trailer and configured to power the one or more ozone generators and the circulation fan.

4. The system of claim 1, wherein the container comprises a human-portable glovebox.

5. The system of claim 1, wherein the receptive region is spaced from the one or more ozone generators by a predefined distance.

6. The system of claim 1, wherein the circulation fan is configured to provide the airflow at a flowrate such that a predefined diffusion time is elapsed before the ozone reaches the receptive region.

7. The system of claim 6, wherein the diffusion time is between five seconds and 90 seconds.

8. The system of claim 1, further comprising a humidifier configured to maintain a humidity in the interior of the container in a predefined humidity range between 75% relative humidity (RH) and 95% RH.

9. The system of claim 1, wherein the one or more ozone generators are configured to effect at least a 3-log reduction in a microbial or viral load on the equipment in response to exposing the equipment to a dose of between 450 parts per million minutes (ppm-min) and 650 ppm-min.

10. The system of claim 1, wherein the one or more ozone generators are configured to effect at least a 6-log reduction in a microbial or viral load on the equipment in response to exposing the equipment to a dose of between 1450 parts per million minutes (ppm-min) and 1550 ppm-min.

11. The system of claim 1, wherein a ratio of a distance between the one or more ozone generators and the receptive region, and a flowrate to which the circulation fan is configured to provide is between five and 90.

12. The system of claim 1, wherein a ratio of a distance between the one or more ozone generators and the receptive region, and a flowrate to which the circulation fan is configured to provide is between 20 and 45.

13. The system of claim 1, further comprising a wire shelf positioned in the interior of the container and configured to receive the equipment.

14. A method for reducing a microbial and/or viral load on equipment using ozone, the method comprising:
(a) positioning the equipment in a receptive region within an interior of a container;
(b) sealing the interior of the container from an environment external the container;
(c) activating one or more ozone generators positioned in the interior of the container to generate ozone; and
(d) operating a circulation fan positioned in the interior of the container to provide an airflow comprising the ozone generated by the one or more ozone generators and directed along a flowpath extending into the receptive region of the interior of the container.

15. The method of claim 14, wherein the equipment comprises personal protective equipment (PPE).

16. The method of claim 14, wherein the container comprises a road-transportable trailer comprising a plurality of wheels.

17. The method of claim 14, wherein (d) comprises effecting at least a 3-log reduction in a microbial or viral load on the equipment in response to exposing the equipment to an ozone dose of between 450 parts per million minutes (ppm-min) and 650 ppm-min.

18. The method of claim 14, wherein (d) comprises effecting at least a 6-log reduction in a microbial or viral load on the equipment in response to exposing the equipment to an ozone dose of between 1450 parts per million minutes (ppm-min) and 1550 ppm-min.

19. The method of claim 14, wherein a ratio of a distance between the one or more ozone generators and the receptive region, and a flowrate to which the circulation fan is configured to provide is between five and 90.

20. The method of claim 14, wherein a ratio of a distance between the one or more ozone generators and the receptive region, and a flowrate to which the circulation fan is configured to provide is between 20 and 45.

21. The method of claim 14, further comprising:
(e) maintaining a humidity in the interior of the container in a predefined humidity range between 75% relative humidity (RH) and 95% RH.
